(19) United States
(12) Patent Application Publication
(10) Pub. No.: US 2016/0257986 A1
(43) Pub. Date: Sep. 8, 2016

(54) Bacterial Diagnosis

(71) Applicant: SRI INTERNATIONAL, Menlo Park, CA (US)

(72) Inventors: Peter B. Madrid, Menlo Park, CA (US); Xiaohe Liu, Menlo Park, CA (US)

(73) Assignee: SRI INTERNATIONAL, Menlo Park, CA (US)

(21) Appl. No.: 15/153,718

(22) Filed: May 12, 2016

Related U.S. Application Data
(63) Continuation of application No. PCT/US14/65308, filed on Nov. 12, 2014.
(60) Provisional application No. 61/902,891, filed on Nov. 12, 2013.

(51) Int. Cl. C12Q 1/04

(52) U.S. Cl. CPC C12Q 1/04

(57) ABSTRACT

Antibiotic susceptibility profile and the bacterial species identification are reported and determined in a single diagnostic test. The method simultaneously reports for a sample of bacteria the identities of the bacteria and their respective antibiotic susceptibilities, the method comprising step(s): contacting the bacteria with antibiotics, a phenotypic fluorescent sensor of antibiotic susceptibility and species- or strain-specific affinity probes, wherein the probes and sensor simultaneously report, respectively, the identities of the bacteria and their respective antibiotic susceptibilities.

### CROSS-REFERENCE TO RELATED APPLICATIONS

This application claims priority to U.S. Ser. No. 61/902,891, filed Nov. 12, 2013, the contents of which is incorporated herein by reference.

### INTRODUCTION

The diagnosis and treatment of bacterial diseases is based initially on symptoms, signs and sometimes imaging (CT scan). Information on the infecting organism and the drug-susceptibility profile for that infection takes over >72 hours. Clinical outcomes are clearly related to the use of appropriate antibiotics to which infecting organism is susceptible. Many deadly bacterial diseases (sepsis, pneumonia, UTIs, etc.) are caused by bacteria that readily acquire new resistance mechanisms.

We disclose a rapid (<3 hours) diagnostic to identify the organisms in an infection and determined phenotypic resistance to a panel of antibiotics—a combination antibiotic susceptibility and bacteria identification test. Our system takes advantage of the fact that bactericidal drugs lead to the generation of reactive oxygen species (ROS) in susceptible bacteria to provide actionable clinical results. The system is also amenable to disclosed novel fluorescent dyes, which are used to sensitively quantitate ROS formation upon exposure to different concentrations of drugs.

### SUMMARY OF THE INVENTION

This invention provides methods and composition for reporting and/or determining the antibiotic susceptibility profile and the bacterial species identification in a single diagnostic test.

In one aspect the invention provides a method of simultaneously reporting for a sample of bacteria the identities of the bacteria and their respective antibiotic susceptibilities, the method comprising step(s): contacting the bacteria with antibiotics, a phenotypic fluorescent sensor of antibiotic susceptibility and species- or strain-specific affinity probes, wherein the probes and sensor simultaneously report, respectively, the identities of the bacteria and their respective antibiotic susceptibilities.

The invention provides embodiments, and all combination thereof, wherein:
the probes are immobilized at discrete loci, with a panel of the loci for each specificity, and the contacting step comprises contacting the bacteria with the probes, and then contacting the probe-bound bacteria of each panel with a corresponding antibiotic and the sensor;
the contacting step comprises contacting the bacteria with the sensor, then dividing bacteria into subsets, then contacting each of the subsets with a corresponding antibiotic and the probes, wherein the probes of each specificity are uniquely labeled;
the sensor reports reactive oxygen species as an indication of antibiotic susceptibility;
the probes are antibodies;
the sensor is dihydrorhodamine 123;
the method is effected in less than 3 hours;
the method determines the antibiotic susceptibility profile and identify of ten pathogenically relevant bacterial species in a single diagnostic test;
the sample is a physiological specimen that is sputum, blood, urine or fecal;
the method further comprising the step of simultaneously detecting probe-bacteria binding and sensor signaling as indicators of the identities of the bacteria and their respective antibiotic susceptibilities, respectively.

The invention also provides kits comprising materials and reagents for, and configured for the subject methods.

The invention specifically provides all combinations of the recited embodiments, as if each had been laboriously individually set forth.

### BRIEF DESCRIPTION OF THE DRAWING

FIG. 1 Solid Phase Embodiment

### DETAILED DESCRIPTION OF PARTICULAR EMBODIMENTS

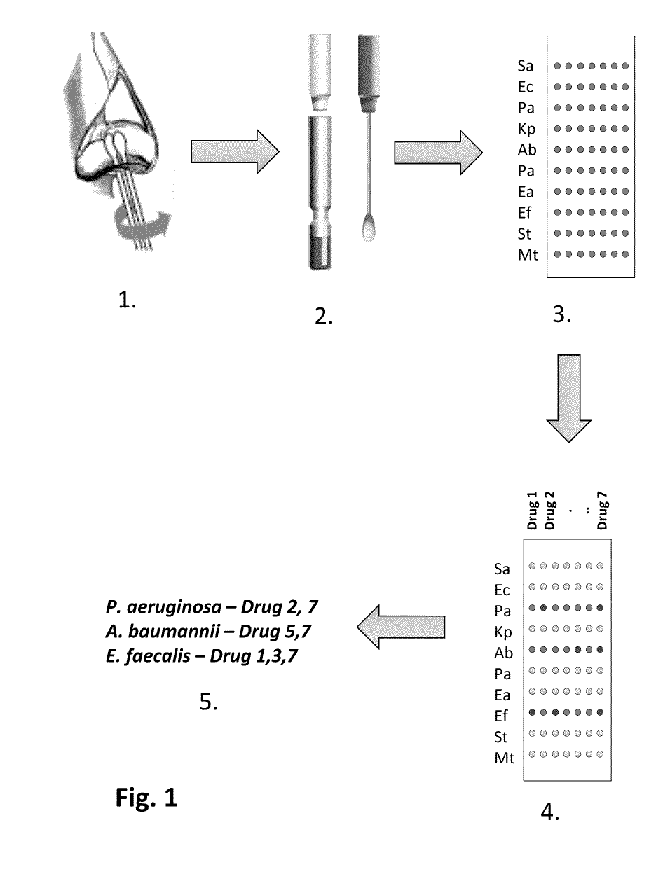

A general protocol is shown in FIG. 1:
1. Collect biological specimen (e.g. sputum, blood, urine, fecal);
2. Transfer to or mix with assay buffer;
3. Incubate with specific capture reagents;
4. Flow drug and ROS detection reagent over specimen; and
5. Read out types of bacteria and susceptibilities.

### EXAMPLES

### Example 1

Cultures of norfloxacin-sensitive E. coli (ATCC 25922; norfloxacin MIC=0.064 μg/ml) were innoculated in MHB liquid media until an OD600 reading of 0.44. The cultures were then diluted with MHB to an OD600 reading of ˜0.1. At time =0 hours, either norfloxacin (final conc. 0.125 μg/ml) or vehicle (DMSO) was added to each culture along with a test experimental sensor dye at 5 μM concentration. The tubes were then incubated in a 37 C incubator with shaking at 1400 rpm. After 1.5 hours the E. coli were analyzed for labeling by flow cytometry using the FITC 488 nm channel. Results showed a significant increase (up to 10-fold) in the treatment groups compared with a vehicle control.

| Compound Identifier | Structure |
|---|---|
| DIBAC (3)4 (positive control) | 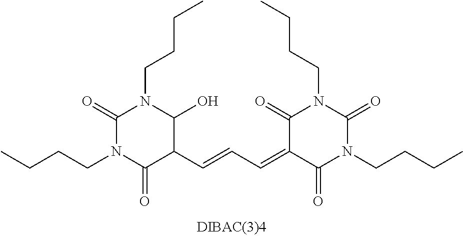 [SMILES: CCCCN1C(=O)C(=C/C=C/C2C(=O)N(CCCC)C(=O)N(CCCC)C2O)C(=O)N(CCCC)C1=O] |
| SRI-009276 | 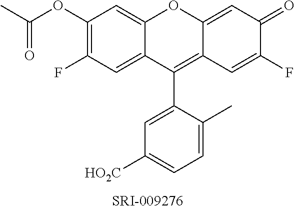 [SMILES: CC(=O)Oc1cc2oc3cc(=O)c(F)cc-3c(-c3cc(C(=O)O)ccc3C)c2cc1F] |
| Dihydrorhodamine 123 | 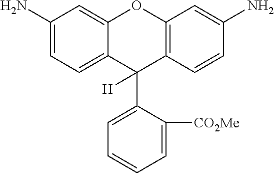 [SMILES: COC(=O)c1ccccc1C1c2ccc(N)cc2Oc2cc(N)ccc21] |

### Example 2

Cultures of norfloxacin-sensitive P. aeruginosa (ATCC 27853; ciprofloxacin MIC=0.25 μg/ml) were inoculated in 3 ml Mueller-Hinton broth (MHB) with an OD600 of 0.081. After 2 hours at 37 C, the cultures were at OD600 of 0.161. At time =0 hours, either ciprofloxacin (final conc. 0.5 μg/ml) or vehicle (water) was added to each culture along with a test experimental sensor dye at 5 μM final concentration. The tubes were then incubated in a 37 C incubator with shaking at 1400 rpm. At 2 hour and 3 hour timepoints, the P. aeruginosa cells were analyzed for labeling by flow cytometry with the FITC 488 nm channel. Both 2 and 3 hour results showed a significant increase (up to 10-fold) in the treatment groups compared with a vehicle control.

| Compound | |
|---|---|
| Identifier | Structure |
| Dihydro- rhodamine 123 | 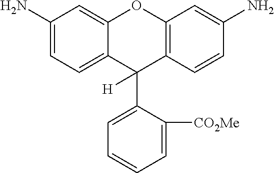 [SMILES: COC(=O)c1ccccc1C1c2ccc(N)cc2Oc2cc(N)ccc21] |
| SRI-006853 | 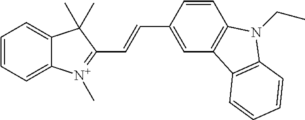 [SMILES: CCn1c2ccccc2c2cc(/C=C/C3=[N+](C)c4ccccc4C3(C)C)ccc21] |
| SRI-009157 | 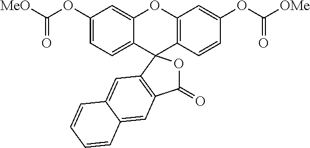 [SMILES: COC(=O)Oc1ccc2c(c1)Oc1cc(OC(=O)OC)ccc1C21OC(=O)c2cc3ccccc3cc21] |

### Example 3

The fluorescent sensors of antibiotic susceptibility work with diverse classes of antibiotics.

Cultures of P. aeruginosa (ATCC 27853) were inoculated in 3 ml Mueller-Hinton broth (MHB) with an OD600 of 0.091. After 2 hours at 37 C, the cultures were at OD600 of 0.179. At time =0 hours, a test antibiotic (cefepime @4 ug/ml, ciprofloxacin @0.05 ug/ml, meropenem @4 ug/ml, aztreonam @8 ug/ml, tobramycin @1 ug/ml) or vehicle (DMSO) was added to each culture along with dihydrorhodamine 123 at 5 μM final concentration. Each antibiotic was tested at twice the concentration of the experimentally determined MIC. The tubes were then incubated in a 37 C incubator with shaking at 1400 rpm. At 2 hour and 3 hour timepoints, the P. aeruginosa cells were analyzed for labeling by flow cytometry with the FITC 488 nm channel. Results showed susceptibility profile for each of the antibiotics.

### Example 4

The use of fluorescent compounds to differentiate drug-sensitive from drug-resistant bacterial strains. General protocol: E. coli sensitive and resistant strains were treated with a reange of norfloxacin concentrations for 2 hours. Results showed differential susceptibility of the strains over the concentration range.

### Example 5

The application of the antibiotic susceptibility methodology to bacterial pathogens including E. coli, P. aeuriginosa, K. pneumonia and S. aureus.

Cultures of P. aeruginosa (ATCC 27853), K. pneumonia, and E. coli were inoculated in 3 ml Mueller-Hinton broth (MHB) with an initial OD600 of 0.147, 0.166 and 0.156 respectively. After 2 hours at 37 C, the cultures were at OD600 of P. aeruginosa-0.161, K. pneumonia-0.604 and E. coli-0.659. To the cultures was then added 75 ul vehicle (water) or 75 ul cipro (2× MIC concentration) followed by 15 ul of DHR. The tubes were then incubated in a 37 C incubator with shaking at 1400 rpm. At 2, 3 and 4 hour timepoints, the cells were analyzed for labeling by flow cytometry with the FITC 488 nm channel Results showed time-course susceptibility profile for each tested bacterial pathogen.

### Example 6

The application of the antibiotic susceptibility testing procedure of Example 5 run concurrently with a marker for bacterial species identification, such as a bacteria-specific antibody.

General Protocol:

1. Add fluorescent sensors of cellular stress;

2. Divide isolate/sample into wells loaded with test antibiotics;

3. Add pathogen identifier probes, differentially labeled; and

4. Analyze marker pattern for each well to determine susceptibility and pathogen identification.

In a single assay we were able to simultaneously measure markers for antibiotic susceptibility (DHR 123) and a marker for species identification (Anti-P. aeruginosa antibody).

Cultures of P. aeruginosa (ATCC 27853) were inoculated in 3 ml Mueller-Hinton broth (MHB) with an OD600 of 0.06. After 2 hours at 37 C, the cultures were at OD600 of 0.150. At time =0 hours, a test antibiotic (ciprofloxacin) or vehicle (DMSO) was added to each culture along with dihydrorhodamine 123 at 5 μM final concentration. At 3 hour and 4 hour timepoints,1 mL of the P. aeruginosa cells were centrifuged and resuspended in FACS buffer with a primary anti-Pseudomonas antibody (rabbit polyclonal Anti-Pseudomonas antibody (ab68538, abcam, Cambridge, Mass.)), and incubated for 15 minutes. The tubes were then centrifuged and resuspended in FACS buffer with a secondary antibody (goat anti-rabbit labeled with Cy7 dye) for another 15 minutes. The samples were then run directly on a FACScaliber flow cytometer for analysis. Results showed a dose-dependent response for antibiotic and for anti-PA antibody.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

1. A method of simultaneously reporting for a sample of bacteria the identities of the bacteria and their respective antibiotic susceptibilities, the method comprising step(s):
contacting the bacteria with antibiotics, a phenotypic fluorescent sensor of antibiotic susceptibility and species- or strain-specific affinity probes, wherein the probes and sensor simultaneously report, respectively, the identities of the bacteria and their respective antibiotic susceptibilities.

2. The method of claim 1 wherein the probes are immobilized at discrete loci, with a panel of the loci for each specificity, and the contacting step comprises contacting the bacteria with the probes, and then contacting the probe-bound bacteria of each panel with a corresponding antibiotic and the sensor.

3. The method of claim 1 wherein the contacting step comprises contacting the bacteria with the sensor, then dividing bacteria into subsets, then contacting each of the subsets with a corresponding antibiotic and the probes, wherein the probes of each specificity are uniquely labeled.

4. The method of claim 1 wherein the sensor reports reactive oxygen species as an indication of antibiotic susceptibility.

5. The method of claim 2 wherein the sensor reports reactive oxygen species as an indication of antibiotic susceptibility.

6. The method of claim 3 wherein the sensor reports reactive oxygen species as an indication of antibiotic susceptibility.

7. The method of claim 1 wherein the probes are antibodies.

8. The method of claim 2 wherein the probes are antibodies.

9. The method of claim 3 wherein the probes are antibodies.

10. The method of claim 1 wherein the sensor is dihydrorhodamine 123.

11. The method of claim 1 effected in less than 3 hours.

12. The method of claim 1 which determines the antibiotic susceptibility profile and identify of ten pathogenically relevant bacterial species in a single diagnostic test.

13. The method of claim 1 wherein the sample is a physiological specimen that is sputum, blood, urine or fecal.

14. The method of claim 1 further comprising the step of simultaneously detecting probe-bacteria binding and sensor signaling as indicators of the identities of the bacteria and their respective antibiotic susceptibilities, respectively.

15. The method of claim 2 further comprising the step of simultaneously detecting probe-bacteria binding and sensor signaling as indicators of the identities of the bacteria and their respective antibiotic susceptibilities, respectively.

16. The method of claim 3 further comprising the step of simultaneously detecting probe-bacteria binding and sensor signaling as indicators of the identities of the bacteria and their respective antibiotic susceptibilities, respectively.

17. The method of claim 1 wherein the sensor reports reactive oxygen species as an indication of antibiotic susceptibility, the probes are antibodies, the sensor is dihydrorhodamine 123, the method is effected in less than 3 hours and determines the antibiotic susceptibility profile and identify of ten pathogenically relevant bacterial species in a single diagnostic test.

18. The method of claim 2 wherein the sensor reports reactive oxygen species as an indication of antibiotic susceptibility, the probes are antibodies, the sensor is dihydrorhodamine 123, the method is effected in less than 3 hours and determines the antibiotic susceptibility profile and identify of ten pathogenically relevant bacterial species in a single diagnostic test.

19. The method of claim 3 wherein the sensor reports reactive oxygen species as an indication of antibiotic susceptibility, the probes are antibodies, the sensor is dihydrorhodamine 123, the method is effected in less than 3 hours and determines the antibiotic susceptibility profile and identify of ten pathogenically relevant bacterial species in a single diagnostic test.

20. The method of claim 1 wherein the sensor reports reactive oxygen species as an indication of antibiotic susceptibility, the probes are antibodies, the sensor is dihydrorhodamine 123, the method is effected in less than 3 hours and determines the antibiotic susceptibility profile and identify of ten pathogenically relevant bacterial species in a single diagnostic test, and further comprising the step of simultaneously detecting probe-bacteria binding and sensor signaling as indicators of the identities of the bacteria and their respective antibiotic susceptibilities, respectively.
